(19) United States
(12) Patent Application Publication
(10) Pub. No.: US 2017/0304054 A1
(43) Pub. Date: Oct. 26, 2017

(54) BONE GEL COMPOSITION AND METHOD OF MANUFACTURE

(71) Applicant: Vivex Biomedical Inc., Marietta, GA (US)

(72) Inventors: Edgar S. Maldonado, Miami, FL (US); Silvia Daniela Gonzales, Miami, FL (US); Wendy W. Weston, Miami, FL (US)

(73) Assignee: Vivex Biomedical, Inc., Marietta, GA (US)

(21) Appl. No.: 15/136,383

(22) Filed: Apr. 22, 2016

(51) Int. Cl. A61F 2/28; A61L 27/36

(52) U.S. Cl. CPC A61F 2/28; A61L 27/3608; A61F 2002/2835; A61F 2310/00359; A61L 2430/02

(57) ABSTRACT

A bone gel composition consists of cortical bone. The cortical bone is made from cut pieces freeze-dried then ground into particles and demineralized then freeze-dried. A volume of the particles is placed in a solution of sterile water to create a mixture, the water volume being twice the second portion, the mixture is autoclaved under heat and pressure to form a gelatin, the resulting bone gel is placed in jars and kept at room temperature or frozen for later use. The cortical bone has the cut pieces having a width, a length and a thickness in the range of 1 to 4 mm The cortical bone pieces are ground to a particle size up to 125 microns.

### TECHNICAL FIELD

This invention is a demineralized bone composition made in the form of a gel. More specifically, a composition that can be formed as a gel or gelatinous composition and a method of manufacture and use of said composition.

### BACKGROUND OF THE INVENTION

The manufacture and use of bone allografts from bone tissue is well known. The use of particles of various specific sizes and distributions have been determined to have beneficial characteristics for new bone growth in the treatment of osseous defects and bone voids.

The issue of getting the repair composition to stay in position has been addressed for various formulations made into malleable paste or putty by the addition of collagen or other gelatinous materials.

The present invention provides an improvement over those prior art materials by providing a bone gel that can be added to autograft or allograft bone particles to make a moldable bone product.

### SUMMARY OF THE INVENTION

A bone gel composition consists of cortical bone. The cortical bone is made from cut pieces freeze-dried then ground into particles and demineralized then freeze-dried. A volume of the particles is placed in a solution of sterile water to create a mixture, the water volume being twice the second portion, the mixture is autoclaved under heat and pressure to form a gelatin, the resulting bone gel is placed in jars and may be kept at room temperature frozen for later use. The cortical bone has the cut pieces having a width, a length and a thickness in the range of 1 to 4 mm The cortical bone pieces are ground to a particle size up to 125 microns.

A method of making a bone gel composition consisting of cortical bone comprises the steps of: preparing cortical bone by cutting the cortical bone into pieces, freeze-drying the pieces and then grinding into particles and demineralizing the ground particles and the freeze-drying the demineralized ground particles to form DBM particles; autoclaving a volume of the DBM particles mixed with sterile water in a 2:1 ratio by volume for a predetermined time at a pre-set temperature and pressure to form a gelatin; cooling the gelatin to form a bone gel; and packaging the resulting bone gel and storing the packaged bone gel.

### Definitions

Cohesiveness is defined as the capacity of DBM aseptic paste to maintain its shape while immersed in normal saline or water for a minimum of one minute.

DBM—Demineralized Bone Matrix.

Cryopreserved—Tissue frozen with the addition of, or in a solution containing, a cryoprotectant agent.

Freeze Dried/Lyophilized—Tissue dehydrated for storage by conversion of the water content of frozen tissue to a gaseous state under vacuum that extracts moisture.

Malleability is the ability of DBM aseptic paste to be molded into different shapes with no visible cracks.

Normal Saline—0.9% Sodium Chloride Solution.

PBS—Phosphate Buffered Saline.

SRI—an equipment sterilization company.

### BRIEF DESCRIPTION OF THE DRAWINGS

The invention will be described by way of example and with reference to the accompanying drawings in which:

FIG. 1 shows a photograph of the bone gel composition in a container and packaged in a clear sealed bag.

FIG. 2 is a photograph of the bone gel composition removed from the packaging with the container lid removed and open.

FIG. 3 is a photograph of the bone gel composition being removed from the container.

FIG. 4 is a photograph of the bone gel composition removed from the container.

FIG. 5 is a photograph of the bone gel composition being worked with.

FIG. 6 is another photograph of the bone gel composition being worked with.

FIG. 7 is a schematic illustration of the bone gel product manufacturing process outline.

FIG. 8 is a schematic illustration of the subprocess of cutting and freeze-drying.

FIG. 9 is a schematic illustration of the subprocess of grinding, demineralization, freeze-drying.

FIG. 10 is a schematic illustration of the subprocess of bone gel preparation.

FIG. 11 is a schematic illustration of the subprocess of packaging.

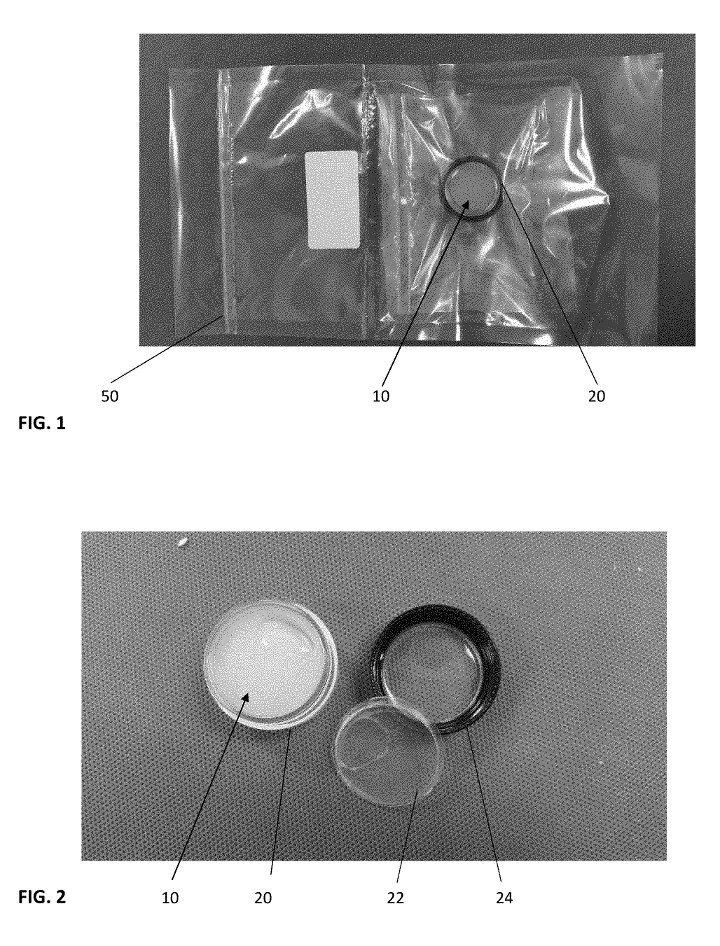

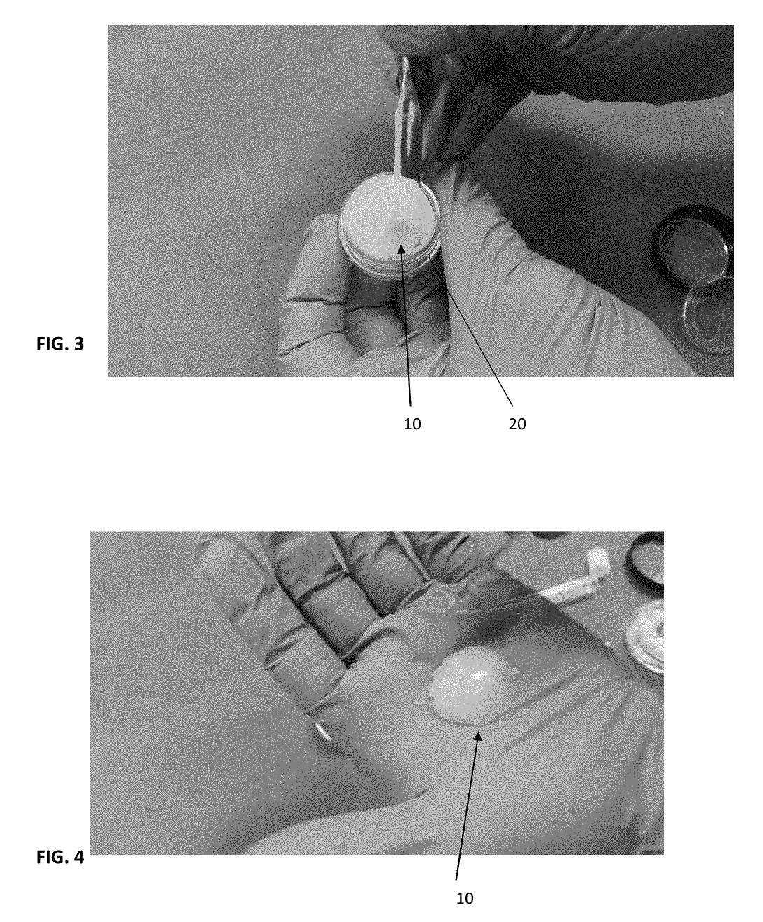

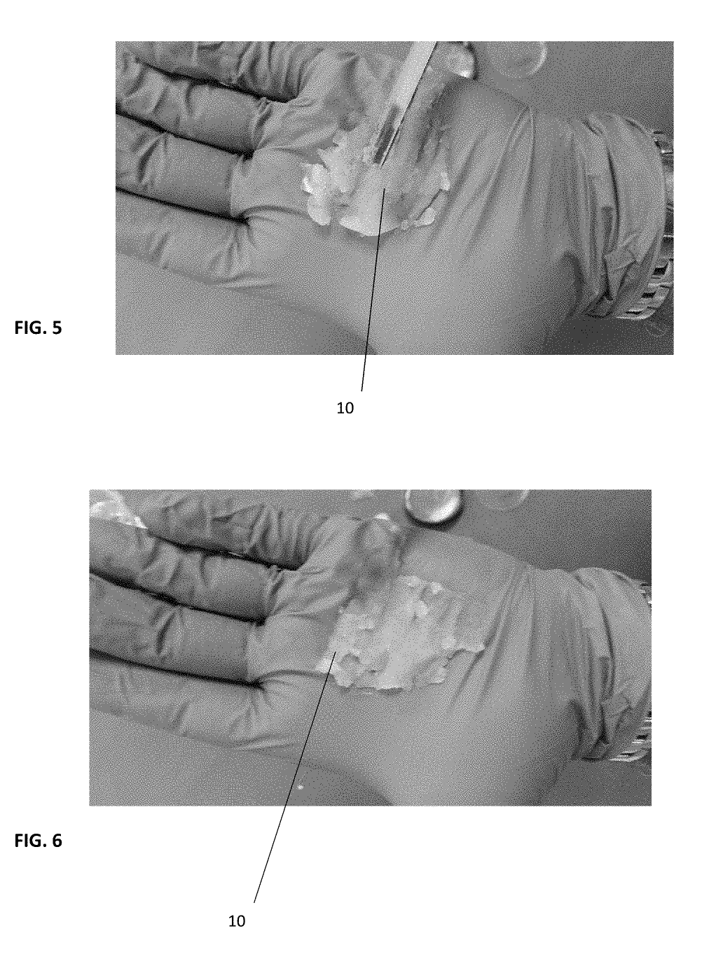

### DETAILED DESCRIPTION OF THE INVENTION

The present invention encompasses the manufacturing of bone gel derived from human cadaveric cortical bone. Cortical bone is obtained from male or female donors within suitable age groups. Full body donors with no joint replacements are preferred. The donors' medical and social history are screened for medical conditions such as osteoporosis and alcohol abuse, which may hinder the intended purpose of the final product. At ambient temperature, bone gel is gelatinous and cohesive. Therefore, it is intended to serve as a binding agent. Bone gel is ready for use or can be mixed with other products.

The bone gel product is entirely derived from cortical bone. The cortical bone is aseptically recovered, cleaned, cut, morselized or shaved, ground, sieved at different sizes, demineralized and freeze-dried to obtain cortical bone particles. Freeze dried, demineralized, ground cortical bone is then mixed with water. The mix is pressurized and heated to form the Bone Gel. Final Bone Gel 10 products of 2cc, 4cc or 8cc are distributed into containers 20, packaged in final packaging 50, as shown in FIG. 1, and stored at room temperature or frozen until distribution to the end user. The amount of bone gel can vary depending on the application.

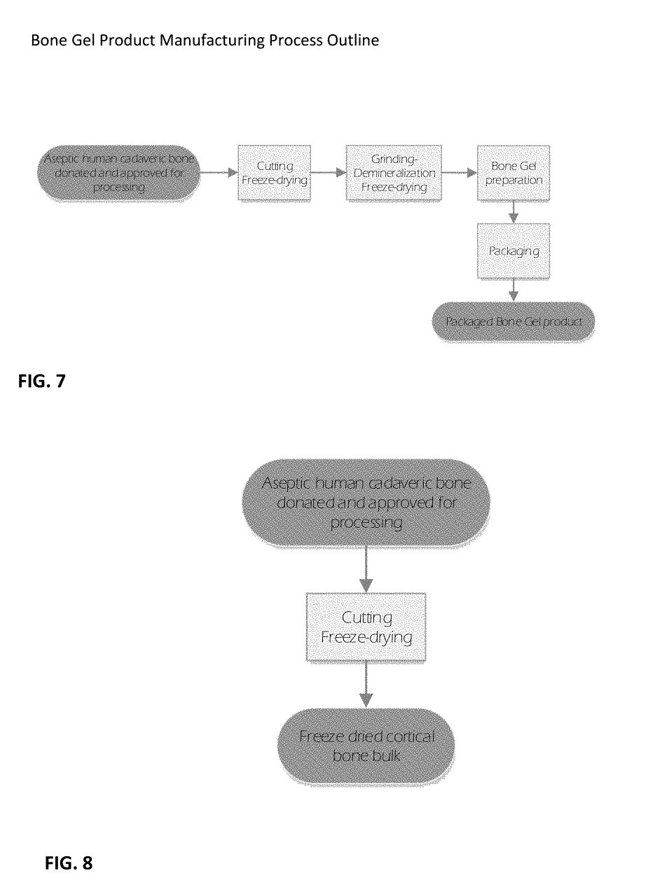

The overall manufacturing process for the Bone gel product can be seen in FIG. 7. The input of the process is the donated and approved for processing aseptic human cadaveric cortical bone immediately frozen after recovery. Once the cortical bone has been processed, the output is the packaged Bone gel product. The process itself has been divided into four subprocesses with their own respective inputs and outputs.

The cutting subprocess is schematically shown in FIG. 8.

Prior to cutting the donated and approved for processing human cadaveric cortical bone, all extraneous material such as muscle fibers, adipose tissue, and periosteum are removed from the tissue. Bones are then rinsed a minimum of 3 times with physiological grade Normal Saline (0.9% Sodium Chloride). Using a band saw, the bones are cut in a manner that the cortical and cancellous portions are separated.

The cortical bone is then cut into small pieces using a band saw. The small pieces are rinsed a minimum of three times in Normal Saline and then placed into a metal container with fresh Normal Saline. The container is aseptically wrapped, placed on a shaker and mechanically agitated for 5 to 10 minutes. The bone tissue is then morselized into 1 to 4 mm length and width pieces, respectively, using a morselizer. The tissue is rinsed again a minimum of three times with Normal Saline in order to remove any remnants of blood and/or fat deposits. The bone pieces are rinsed with hydrogen peroxide for no more than 10 minutes to remove fat/blood. The bone pieces are rinsed a minimum of three times with sterile water to remove any residual hydrogen peroxide. Then, the bone tissue is placed in a metal container and stored at −80° C. The frozen bone tissue is freeze dried with a cycle set for 33 hours and 50 minutes. It is understood the timing, ratios and volumes can vary based on the equipment and procedures used and the above is exemplary of the preferred process for the inventors' equipment.

The Grinding-Demineralization subprocess is shown in FIG. 9.

Once the freeze drying cycle is completed, the cortical bulk is ground and sieved to obtain particle sizes of up to 125 um, typically 25 to 125 um. The particulate tissue is demineralized by addition of 0.6 HCL solution at a 20:1 ratio (20 ml of 0.6 HCL to 1 g of bone). The solution containing the tissue is placed on a magnetic stir plate for 19 minutes. After decanting the liquid, the particulate tissue is mixed with sterile water at a 20:1 ratio (20 ml of sterile water to 1 g of bone). The solution containing the tissue is placed on a magnetic stir plate for 4 minutes. The process of decanting, mixing and incubating for 4 minutes is repeated with PBS solution. After decanting the PBS, the particulate tissue is mixed with sterile water at a 20:1 ratio (20 ml of sterile water to 1 g of bone). The solution containing the tissue is placed on a magnetic stir plate for 9 minutes. The water waste solution is decanted and the demineralized particulate tissue is stored at −80° C. The frozen, demineralized particulate tissue is freeze dried for 33 hours 50 minutes. At the end of the freeze drying process, samples can be collected for residual moisture and residual calcium testing. It is understood the timing, ratios and volumes can vary based on the equipment and procedures used and the above is exemplary of the preferred process for the inventors' equipment.

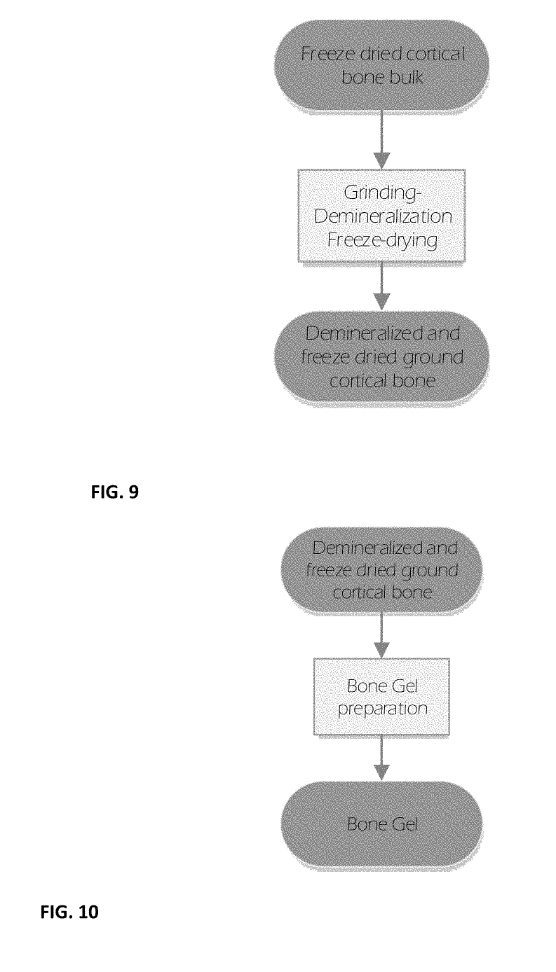

The Bone Gel Preparation subprocess is shown in FIG. 10.

The demineralized, freeze-dried cortical bone particles can be divided into groups of 100 cc, approximately. Each group is placed in a Pyrex glass bottle and mixed with sterile water. The ratio of sterile water to particles is 2:1 by volume. In order to prepare Bone Gel, the mix is autoclaved for 1.25 hours. The autoclaving process includes conditioning (15 minutes), exposure (30 minutes) and drying (30 minutes). Temperature during the exposure step is 121.1° C. and the pressure is 30.15 psi. After autoclaving is completed, the Bone Gel is aliquoted to jars. It is understood the timing, ratios and volumes can vary based on the equipment and procedures used and the above is exemplary of the preferred process for the inventors' equipment.

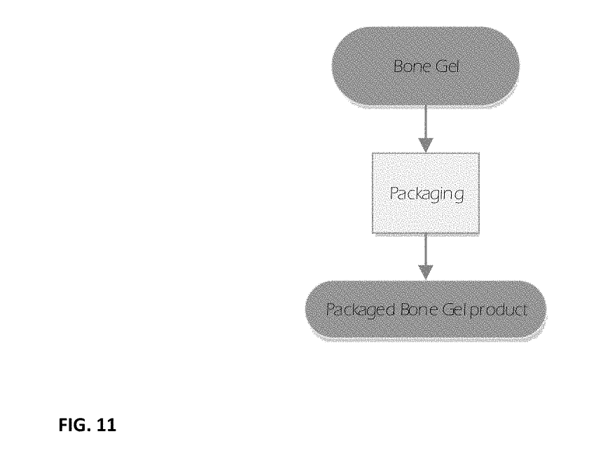

The Packaging subprocess is shown in FIG. 11.

The Bone Gel is packaged in closed containers. The containers have a lid insert 22 secured by a cap 24. The packaged final products are stored at room temperature or frozen until they are distributed to the end user. Batch release is contingent upon final culture results.

The bone gel composition is designed to be mixed with allograft or autograft bone particles, strands, growth factors, bone putty, bone paste, cells, or other bone growth enhancing products.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims.

What is claimed is:

1. A bone gel composition consisting of cortical bone consists of cortical bone made from cut pieces freeze dried then ground into particles and demineralized then freeze-dried, wherein a volume of the particles is placed in a solution of sterile water to create a mixture, the water volume being twice the second portion, the mixture is autoclaved under heat and pressure to form a gelatin, the resulting bone gel is placed in jars for later use.

2. The bone gel composition of claim 1 wherein the cortical bone has the cut pieces having a width, a length and a thickness in the range of 1 to 4 mm.

3. The bone gel composition of claim 2 wherein the cortical bone pieces are ground to a particle size up to 125 microns.

4. The bone gel composition of claiml wherein the bone gel when placed in jars is then frozen.

5. A method of making a bone gel composition consisting of cortical bone comprises the steps of:
preparing cortical bone by cutting the cortical bone into pieces, freeze-drying the pieces and then grinding into particles and demineralizing the ground particles and the freeze-drying the demineralized ground particles to form DBM particles;
autoclaving a volume of the DBM particles mixed with sterile water in a 2:1 ratio by volume for a predetermined time at a pre-set temperature and pressure to form a gelatin;
cooling the gelatin to form a bone gel; and
packaging the resulting bone gel.

6. The method of making a bone gel composition of claim 5 wherein the cortical bone has the cut pieces having a width, a length and a thickness in the range of 1 to 4 mm

7. The method of making a bone gel composition of claim 5 wherein the cortical bone pieces are ground to a particle size up to 125 microns.

8. The method of making a bone gel composition of claim 5 includes the step of freezing the bone gel after packaging.
